(19) United States
(12) Patent Application Publication
(10) Pub. No.: US 2010/0209081 A1
(43) Pub. Date: Aug. 19, 2010

(54) Electrical Device for Emanating a Volatile Liquid

(75) Inventor: Shaun Rymer, Hull (GB)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: Reckitt & Colman (Overseas) Limited, Slough, Berkshire (GB)

(21) Appl. No.: 12/680,858

(22) PCT Filed: Oct. 1, 2008
(86) PCT No.: PCT/GB08/03304

§ 371 Date: Mar. 30, 2010

(30) Foreign Application Priority Data
Oct. 5, 2007 (GB) 0719510.0

(51) Int. Cl. A61L 9/03

(52) U.S. Cl. 392/395

(57) ABSTRACT

A device for evaporating a volatile liquid from a refill, the device comprising: a housing;

securing means within said housing to releasably secure the refill to the device;

electrical heating means within said housing; and

chimney means within said housing having a lower aperture and an upper aperture to define a channel therebetween to the exterior of the device;

wherein the refill for use with the device comprises:

a reservoir portion for holding a quantity of volatile liquid; and

a wick having a proximal end within the reservoir portion substantially adjacent a base of said reservoir portion and having a distal end extending above the reservoir portion;

wherein the device is configured such that, in use, the securing means are adapted to hold the refill such that the distal end of the wick is located within the chimney means;

characterised in that the chimney means are rotatable relative to the housing and said means have a continuous wall between the lower and upper apertures wherein said wall is of a varying thickness.

### FIELD OF THE INVENTION

This invention relates to a device for emanating volatile liquids, for example air fresheners and insecticides or the like. The invention relates in particular to a device for emanating volatile liquids from a container into a room, with the assistance of electrical power.

### BACKGROUND

Electrical devices for emanating volatile liquid from a refill are known. Typically a refill consists of a bottle or the like that houses a volume of volatile liquid with a sealed closure member to retain the liquid within the bottle and a wick of a length such that one end is disposed within the liquid and the other end extends through the closure member and projects above the bottle.

The known electrical devices are shaped to permit the free end of the wick (i.e. the part of the wick projecting above the rest of the bottle) to sit within a chamber of the device that is open at an upper end to permit the emanation of the liquid therethrough. The chamber of the device also contains a heater located therein or nearby to accelerate the evaporation of volatile liquid from the wick. The bottle, wick and heater are normally retained within a casing which carries an electric plug. To operate the heater the device is plugged into a wall socket to cause a flow of electricity to the heater.

Some of the known devices purport to offer control of the rate of evaporation of the volatile liquids. In one device, described in Spanish patent application No. 9701388, the rate of evaporation is altered by varying the relative position of the wick and the heater (which typically is ring-shaped). This known device contains a means for moving the container and the wick axially, through the action of a screw thread, whilst the ring heater is kept stationary.

In another known device, the relative movement of a ring heater and a wick is achieved by keeping the wick stationary and moving the heater axially.

In another device on the market, a tiltable barrel device is located at the distal end of the wick. This may be tilted about a horizontal axis to alter the air flow pathways at the distal end of the wick, and thereby alter the rate of evaporation.

However, such known devices are often found to be unsatisfactory in giving a good range of adjustment of heat applied to the wick. Indeed, testing of known devices suggests that the rate of evaporation of volatile liquids is somewhat arbitrary as some of the known devices produce a higher rate of evaporation of the volatile liquid when they are at their minimum setting than when at their supposed maximum setting.

It is an object of the present invention to overcome, or at least mitigate, the above-mentioned problems by providing a device which enables improved regulation of the rate of evaporation of volatile liquids.

### SUMMARY OF INVENTION

According to a first aspect of the present invention there is provided therefore a device for evaporating a volatile liquid from a refill, the device comprising:

a housing;

securing means within said housing to releasably secure the refill to the device;

electrical heating means within said housing; and

chimney means within said housing having a lower aperture and an upper aperture to define a channel therebetween to the exterior of the device;

wherein the refill for use with the device comprises:

a reservoir portion for holding a quantity of volatile liquid; and

a wick having a proximal end within the reservoir portion substantially adjacent a base of said reservoir portion and having a distal end extending above the reservoir portion;

wherein the device is configured such that, in use, the securing means are adapted to hold the refill such that the distal end of the wick is located within the chimney means;

characterised in that the chimney means are rotatable relative to the housing and said means have a continuous wall between the lower and upper apertures wherein said walls are of a varying thickness.

Preferably the wick of the refill to be used with the device of the present invention is substantially circular in cross-section, wherein said cross-section is taken substantially perpendicular to the longitudinal axis of the wick.

In a preferred first embodiment the exterior surface of the chimney means may be substantially circular in cross-section, wherein said cross-section is taken substantially perpendicular to the longitudinal axis of said means. The interior surface of the chimney means in this preferred first embodiment may also be substantially circular in cross-section, wherein said cross-section is taken substantially perpendicular to the longitudinal axis of said means. This preferred first embodiment being characterised in that the central longitudinal axis of said interior surface may be parallel to but offset from the central longitudinal axis of said exterior surface.

Preferably the wick and the exterior surface of the chimney means are longitudinally co-axial, whereas the longitudinal axis of the interior surface is parallel to but not co-axial therewith.

In an alternatively preferred first embodiment the interior surface of the chimney means may have a cross-section that is curved with a continually increasing radius. In this increasing radius embodiment the beginning of the curve (having the smaller radius) may be joined to the end of the curve (having the larger radius) by a radially straight line.

As a further alternatively preferred first embodiment, the interior surface of the chimney means may be shaped such that at least one portion of the chimney walls is thicker between the interior and exterior surfaces than at least one other portion of the chimney walls.

In a preferred second embodiment the exterior surface of the chimney means may be substantially circular in cross-section, wherein said cross-section is taken substantially perpendicular to the longitudinal axis of said means. The interior surface of the chimney means in this preferred second embodiment may also be substantially circular in cross-section, wherein said cross-section is taken substantially perpendicular to the longitudinal axis of said means. This preferred second embodiment being characterised in that the central longitudinal axis of said exterior surface may be parallel to but offset from the central longitudinal axis of said interior surface.

Preferably the wick and the interior surface of the chimney means are longitudinally co-axial, whereas the longitudinal axis of the exterior surface is parallel to but not co-axial therewith.

In an alternatively preferred second embodiment the exterior surface of the chimney means may have a cross-section that is curved with a continually increasing radius. In this increasing radius embodiment the beginning of the curve (having the smaller radius) may be joined to the end of the curve (having the larger radius) by a radially straight line.

As a further alternatively preferred second embodiment, the exterior surface of the chimney means may be shaped such that at least one portion of the chimney walls is thicker between the interior and exterior surfaces than at least one other portion of the chimney walls.

With any of the aforesaid arrangements of interior surface(s) of the chimney means, said surface(s) is preferably sized/shaped such that it/they does not make direct contact, in use, with the wick of the refill.

The heating means is located within the housing and located adjacent the chimney means. The ability of the chimney means to be rotated by a user permits a user to rotate the chimney means relative to the heating means, thus altering the thickness of the chimney walls between the heating means and, in use, the wick of the refill.

The chimney means is preferably constructed from a plastics material.

For the preferred first embodiment, surprisingly, in use, when the chimney means of plastics material are rotated such that the thickness of the chimney wall between the heating means and the wick is increased, the heat applied to the wick is increased. Conversely, in use, when the chimney means are rotated such that the thickness of the chimney wall between the heating means and the wick is decreased, the heat applied to the wick is decreased.

Without wishing to be bound by theory, it is hypothesised that an increase in the thickness of the chimney wall between the heating means and, in use, the wick results in a small air gap between the interior surface of the chimney means and the wick, thus less heat insulation of the wick by the gap. Equally, a decrease in the thickness of the chimney wall between the heating means and, in use, the wick results in a larger air gap between the interior surface of the chimney means and the wick, thus greater heat insulation of the wick.

For the preferred second embodiment, surprisingly, in use, when the chimney means of plastics material are rotated such that the thickness of the chimney wall between the heating means and the wick is increased, the heat applied to the wick is decreased. Conversely, in use, when the chimney means are rotated such that the thickness of the chimney wall between the heating means and the wick is decreased, the heat applied to the wick is increased.

Without wishing to be bound by theory, it is hypothesised that an increase in the thickness of the chimney wall between the heating means and, in use, the wick results in greater heat insulation of the wick. Equally, a decrease in the thickness of the chimney wall between the heating means and, in use, the wick results a reduction of the heat insulation of the wick.

Regardless of the mode of operation, it has been surprisingly found that the chimney means of the present invention can facilitate the production of a device for emanating volatile liquids with hitherto unforeseen performance offering variable control of the heat applied, in use, to a wick of a refill.

The cross-sectional thickness of the chimney wall varies between a thicker to thinner portion of the wall in a ratio of 3:2-10:1, and preferably in a ratio of between 2:1-8:1, and most preferably in a ratio of between 2:1-6:1.

The chimney means are preferably continuous. The chimney means may by made as a one-piece component. Preferably the thickness of the wall of the chimney is longitudinally uniform.

As mentioned, the chimney means is preferably made from a plastics material, and preferably from a plastics material that is a good thermal conductor such as nylon or polypropylene. Alternatively, the chimney means may be made from a metal, metalised material, an alloy and/or combination thereof.

Alternatively, the chimney means may be made from a material that is a good thermal insulator, such as glass or certain ceramic materials.

Again, without wishing to be bound by theory, it is hypothesised that where the chimney means is made from a material having a high thermal insulation properties in the preferred first embodiment, an increase in the thickness of the chimney wall between the heating means and, in use, the wick results in a greater amount of heat insulation of the wick and a lower temperature thereof despite the small air gap between the interior surface of the chimney means and the wick. Conversely, a decrease in the thickness of the chimney wall between the heating means and, in use, the wick results in less heat insulation of the wick despite the larger air gap between the interior surface of the chimney means and the wick, thus a greater temperature of the wick.

Similarly, it is hypothesised that where the chimney means is made from a material having a high thermal insulation properties in the preferred second embodiment, an increase in the thickness of the chimney wall between the heating means and, in use, the wick results in a greater amount of heat insulation of the wick and a lower temperature thereof. Conversely, a decrease in the thickness of the chimney wall between the heating means and the wick, in use, results in less heat insulation of the wick, thus a greater temperature of the wick.

Preferably the heating means is fixed in position within the device such that it remains at a predetermined distance from the chimney means.

Alternatively, the heating means may be laterally movable toward and away from the chimney means in coordination with the rotation of the chimney means which may increase the performance of the device. Specifically, it is envisaged that the heating means may be operable to move toward the chimney means when the chimney means is rotated toward its particular position for maximum heat transfer from the heating means to the wick. Conversely, when the chimney means is rotated for a reduced amount of heat transfer from the heating means to the wick the heating means may move further away from the chimney.

The heating means may be provided in any suitable form such as a bar heater or at least one point source resistor. Preferably the heating means is provided in the form of at least one electrical resistor, such as a thermistor. Preferably, at least one electrical resistor is used in the device of the present invention as the heating means.

The electrical power applied to the device may be provided by any suitable form, such as by mains electricity, batteries or solar cells. However, due to the power demands of the heating means, mains electricity is preferred.

Where the device is intended for use with mains electricity, the device may be provided with plug formations configured to engage the openings in a mains electrical socket.

Where the device is provided with the plug formations, the formations may be located toward a generally rear-facing aspect of the housing and, in this arrangement, the securing means can be located in a lower-facing aspect of the housing and the upper aperture of the chimney means can be located in an upper aspect of the housing.

The device may be provided with control means that directly or indirectly engage or communicate with or are integral with the rotatable chimney means to permit a user of the device to rotate the chimney means in order to alter, in use, the temperature applied from the heating means to the wick of the refill, thus altering the rate of emanation of the volatile liquid into the environment surrounding the device.

The housing is preferably substantially open toward the lower aspect thereof to permit convenient access to, and sight of, a refill when releasably held by the securing means. This arrangement may be advantageous since a user will be able to visually monitor the level of volatile liquid remaining in the refill.

Alternatively, the housing may substantially surround the refill when the refill is held by the securing means. This arrangement may be advantageous as an engaged refill may be less susceptible to being tampered with. Additionally, this arrangement may permit the device to have an improved aesthetic from a consumer perspective.

The securing means may engage any part of the refill to ensure the position of the refill in relation to the device. Preferably the securing means are arranged to engage with an upper portion of the refill since this arrangement may facilitate a more reliable positioning of the wick within the chimney means.

Alternatively, the securing means may engage a lower portion of the refill. This arrangement may be particularly useful where the housing is arranged to substantially surround an engaged refill.

The securing means may be arranged to engage more than one portion of the refill.

The volatile liquid may be provided in the form of an air freshener, a deodorant, a perfume, an odourant, an insecticide, a fungicide and/or variants thereof.

According to a second aspect of the present invention there is provided therefore a device for evaporating a volatile liquid, the device comprising:

a refill comprising a reservoir portion for holding a quantity of volatile liquid and a wick having a proximal end within the reservoir portion substantially adjacent a base of said reservoir portion and having a distal end extending above the reservoir portion;

a housing;

electrical heating means within said housing;

chimney means within said housing having a lower aperture and an upper aperture to define a channel therebetween to the exterior of the device; and

securing means within said housing to releasably secure the refill to the device such that the distal end of the wick is located within the chimney means;

characterised in that the chimney means are rotatable relative to the housing and said means have a continuous wall between the lower and upper apertures wherein said wall is of a varying thickness.

According to a third aspect of the present invention there is provided therefore a method of varying the rate of emanation of a quantity of volatile liquid, wherein the method comprises:

engaging a refill with a device according to the first aspect of the present invention;

said refill comprising a reservoir portion for holding a quantity of volatile liquid and a wick having a proximal end within the reservoir portion substantially adjacent a base of said reservoir portion and having a distal end extending above the reservoir portion;

applying electrical power to the device;

effecting rotation of the chimney means to alter the heat applied to said wick.

### BRIEF DESCRIPTION OF THE DRAWINGS

Embodiments of the invention will now be described, by way of example only, with reference to the following drawings in which:

FIG. 1 illustrates a front view of a device in accordance with the present invention;

FIG. 2 illustrates a plan view of the device in accordance with the present invention;

FIG. 3 illustrates a plan view of a chimney means according to a preferred first embodiment of the present invention;

FIG. 4 illustrates a plan view of a chimney means according to an alternative preferred first embodiment of the present invention;

FIG. 5 illustrates a plan view of a chimney means according to a preferred second embodiment of the present invention; and

FIG. 6 illustrates a plan view of a chimney means according to an alternative preferred second embodiment of the present invention.

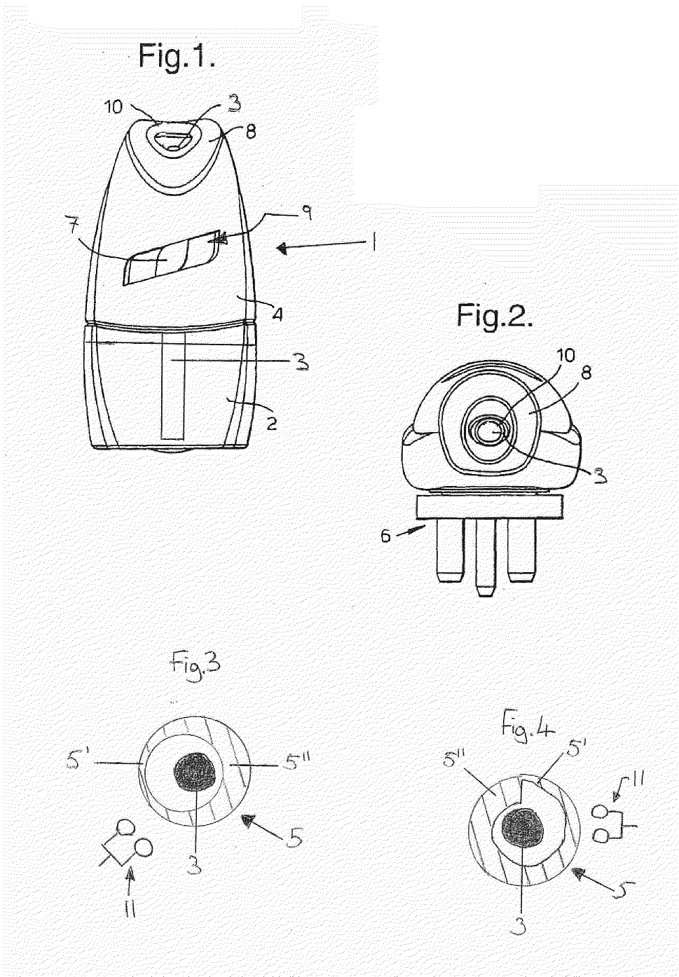

In general terms, the device 1 illustrated in FIGS. 1 & 2 is shown with a refill engaged therewith. The refill has a reservoir portion in the form of a glass bottle 2 containing a volatile liquid and a wick 3 extending into the bottle 2. The wick 3 extends above the top of the bottle (not shown) through a seal (not shown) and into a chimney means 5 of the device. The wick 3 may be substantially cylindrical. The seal is present to retain the liquid within the bottle 2 should the device 1 be knocked over and/or inverted when the refill is engaged therewith.

The device 1 has a plastics housing 4 which partially extends over the refill and its upper part. From the rear wall of the housing extends an electrical plug 6. In this embodiment it is a British-style three-pin plug but for other countries the appropriate plug may be employed; such as a two-pin plug.

The top of the housing is, for aesthetic reasons, somewhat downwardly inclined in the forward direction such that the housing terminates in a discrete slanted top face 8. Within the slanted top face there is a generally circular central aperture 10. Aperture 10 defines the upper aperture of the chimney means. The upper aperture 10 is aligned with a co-axial lower aperture (not shown), thus defining a channel therebetween for volatile liquid to flow up and out of the upper aperture into the environment surrounding the device 1.

Referring to FIGS. 1-4 now, within the housing 4 is contained the chimney means 5 and the heating means 11. In FIGS. 3-4, the heating means are represented as a pair of resistors 11, which may be provided in the form of positive temperature coefficient (PTC) thermistors. However, the heating means could be provided by way of a ring heater or the like.

When a refill is engaged with the device 1, the wick 3 enters the channel within the chimney means 5. In one embodiment shown in FIG. 3, when the wick 3 is positioned within the channel, the wick 3 is longitudinal co-axial with the exterior surface of the chimney, the interior surface having a parallel but non-common longitudinal axis.

However, in an alternative embodiment as shown in FIG. 4, when the wick 3 is positioned within the channel, the wick 3 is longitudinal co-axial with the chimney as a whole, the interior surface thereof having a continually increasing radius.

In both embodiments, the chimney means 5 has a varying wall thickness, from a thinner wall portion 5′ to a thicker wall portion 5″. While the thickness of the wall varies around the circumference of the chimney 5, the distance between the wick 3 and the exterior surface of the chimney 5 and the thermistors 11 all remain static. What does vary however is the distance between the wick 3 and the interior surface of the chimney 5 and the associated air gap therebetween relative to the heating means 11. The varying distance results in a varying air gap which varies the amount of heat insulated or not from the surface of the wick 3.

Where the chimney 5 is made from a plastics material, the smaller the air gap the greater the amount of heat that is passed to the wick 3 and, thus, the greater the rate of emanation of the volatile liquid.

The device 1 is illustrated as being provided with control means 9. The control means 9 has a switch 7 that allows a user to control the rate of emanation of the volatile liquid by controlling the heat applied from the thermistors 11 to the wick 3 in the chimney 5. Specifically, the switch 7 may be operable to directly or indirectly engage the chimney 5 to rotate it between: (i) the wall 5′ being between the wick and the thermistors 11; to (ii) the wall 5′ being between the wick 3 and the thermistors 11.

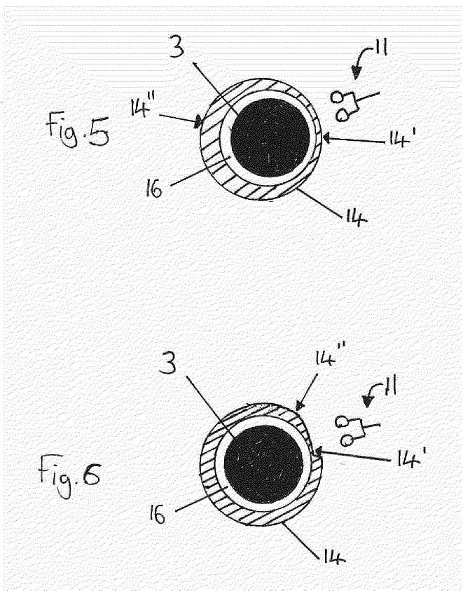

In FIGS. 5-6, the heating means are again represented as a pair of resistors 11, which may be provided in the form of positive temperature coefficient (PTC) thermistors located adjacent to but spaced from the chimney means 14.

When a refill is engaged with the device 1, the wick 3 enters the channel within the chimney means 14. In FIGS. 5 the wick 3 is positioned within the channel 16 and is substantially longitudinal co-axial with the interior surface of the chimney 14 such that the airspace gap between the exterior surface of the wick 3 and the interior surface of the chimney means is substantially constant. The exterior surface of the chimney means 14 has a parallel but non-common longitudinal axis with the wick 3.

In an alternative embodiment as shown in FIG. 6, the wick 3 is again positioned within the channel 16 and is substantially longitudinal co-axial with the interior surface of the chimney 14 such that the airspace gap between the exterior surface of the wick 3 and the interior surface of the chimney means is substantially constant. The wick 3 is also longitudinal co-axial with the chimney as a whole, the exterior surface of said chimney means 14 having a continually increasing radius over at least a portion thereof.

In both FIGS. 5-6 the chimney means 14 has a varying wall thickness, from a thinner wall portion 14′ to a thicker wall portion 14″. While the thickness of the wall varies around the circumference of the chimney 14, the distance between the wick 3 and the interior surface of the chimney 14 and the thermistors 11 all remain static. What does vary however is the distance between the wick 3 and the exterior surface of the chimney 14 and the associated thermal insulation therebetween relative to the heating means 11. The varying distance results in a varying chimney wall thickness which varies the amount of heat insulated or not from the surface of the wick 3.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A device adapted to evaporate a volatile liquid from a refill, the device comprising:
a housing;
securing means within said housing to releasably secure the refill to the device;
electrical heating means within said housing; and
chimney means within said housing has a lower aperture and an upper aperture to define a channel therebetween to the exterior of the device;
wherein the refill for use with the device comprises:
a reservoir portion for holding a quantity of volatile liquid; and
a wick having a proximal end within the reservoir portion substantially adjacent a base of said reservoir portion and having a distal end extending above the reservoir portion;
wherein the device is configured such that, in use, the securing means are adapted to hold the refill such that the distal end of the wick is located within the chimney means;
wherein the chimney means are rotatable relative to the housing and said means have a continuous wall between the lower and upper apertures wherein said walls are of a varying thickness.

2. A device according to claim 1, wherein the chimney means further includes an exterior surface having a central longitudinal axis, and an interior surface having a central longitudinal axis, wherein the exterior surface of the chimney means is substantially circular in cross-section and the interior surface of the chimney means is substantially circular in cross-section, and wherein the central longitudinal axis of said interior surface is parallel to but offset from the central longitudinal axis of said exterior surface.

3. A device according to claim 2, wherein the interior surface of the chimney means has a cross-section that is curved with a continually increasing radius.

4. A device according to claim 3, wherein the beginning of the curve having the smaller radius is joined to the end of the curve having the larger radius by a radially straight line.

5. A device according to claim 2, wherein the interior surface of the chimney means is shaped such that at least one portion of the chimney means is thicker between the interior and exterior surfaces than at least one other portion of the chimney means.

6. A device according to claim 2, wherein the interior surface of the chimney means is substantially circular in cross-section and the exterior surface of the chimney means is substantially circular in cross-section, and wherein the central longitudinal axis of said exterior surface is parallel to but offset from the central longitudinal axis of said interior surface.

7. A device according to claim 2, wherein the exterior surface of the chimney means has a cross-section that is curved with a continually increasing radius.

8. A device according to claim 7, wherein the beginning of the curve having the smaller radius is joined to the end of the curve having the larger radius by a radially straight line.

9. A device according to claim 2, wherein the exterior surface of the chimney means is shaped such that at least one portion of the chimney means is thicker between the interior and exterior surfaces than at least one other portion of the chimney means.

10. A device according to claim 2, wherein interior surface is sized/shaped such that it does not make direct contact, in use, with the wick of the refill.

11. A device according claim 2, wherein the cross-sectional thickness of the chimney wall varies between a thicker to thinner portion of the wall in a ratio of 3:2-10:1.

12. A device according to claim 1, wherein the chimney means is made from a plastics material.

13. A device according to claim 1, wherein control means are provided to permit a user of the device to rotate the chimney means.

14. A device adapted to evaporate a volatile liquid, the device comprising:
a refill comprising a reservoir portion for holding a quantity of volatile liquid and a wick having a proximal end within the reservoir portion substantially adjacent a base of said reservoir portion and having a distal end extending above the reservoir portion;
a housing;
electrical heating means within said housing;
chimney means within said housing having a lower aperture and an upper aperture to define a channel therebetween to the exterior of the device; and
securing means within said housing to releasably secure the refill to the device such that the distal end of the wick is located within the chimney means;
wherein the chimney means are rotatable relative to the housing and said means have a continuous wall between the lower and upper apertures wherein said walls are of a varying thickness.

15. A method of varying the rate of emanation of a quantity of volatile liquid, wherein the method comprises:
engaging a refill with a device according to claim 1,
said refill comprising a reservoir portion for holding a quantity of volatile liquid and a wick having a proximal end within the reservoir portion substantially adjacent a base of said reservoir portion and having a distal end extending above the reservoir portion;
applying electrical power to the device;
effecting rotation of the chimney means to alter the heat applied to said wick.

16. (canceled)

17. (canceled)
